(19) United States
(12) Patent Application Publication
(10) Pub. No.: US 2014/0024546 A1
(43) Pub. Date: Jan. 23, 2014

(54) SYSTEMS AND METHODS FOR NORMALIZING GENE EXPRESSION PROFILES OF BIOLOGICAL SAMPLES HAVING A MIXED CELL POPULATION

(71) Applicant: SYNAPDX CORPORATION, (US)

(72) Inventor: Stanley Letovsky, Milton, MA (US)

(73) Assignee: SYNAPDX CORPORATION, Lexington, MA (US)

(21) Appl. No.: 13/875,257

(22) Filed: May 1, 2013

Related U.S. Application Data
(60) Provisional application No. 61/641,253, filed on May 1, 2012.

(51) Int. Cl. C12Q 1/68

(52) U.S. Cl. CPC C12Q 1/6813
USPC 506/9; 435/6.12; 435/287.2; 506/39

(57) ABSTRACT

The present disclosure generally provides methods for normalizing gene expression profiles against a cellular repertoire, i.e., the different proportions of various cell types in a sample containing multiple cell types. More specifically, the present disclosure encompasses a method for normalizing the gene expression profile against a mixed cell population.

### RELATED APPLICATIONS

This application claims priority to and the benefit of, and incorporates herein by reference in its entirety, U.S. Provisional Patent Application No. 61/641,253, “Normalizing Blood Gene Expression Profiles,” filed May 1, 2012.

### FIELD

The present disclosure relates to methods for normalizing gene expression profiles, for example, for purposes of disease diagnosis.

### BACKGROUND

Gene expression profiling is a genomic technique for measuring activity (expression) of many genes in a cell at a particular point in time. The complement of mRNA in a cell largely dictates its complement of proteins. Consequently, gene expression is a major determinant of the biology of both normal and diseased cells. In medical diagnostics, gene expression profiles, e.g., quantitative RNA levels, in specific tissues of the body may be used to diagnose or classify diseases.

Gene expression data from blood genomic studies is a powerful tool for studying human disease. Blood is a mixture of multiple cell types, including neutrophils, eosinophils, basophils, lymphocytes, monocytes, T cells, B cells, and NK cells. Blood also includes subpopulations of these cell types, identified by the presence or absence of certain markers, such as CD4. Information relevant to the diagnosis of disease may be obtained from the counts or percentages of these types of cells in a particular sample, as well as from gene expression data. The predictive gene expression signatures that are used for diagnosis can be weakly expressed and highly variable, however. The identification of these signatures can be problematic due to the fact that blood is a mixed tissue, comprising multiple types of cells, so that differential expression profiles can reflect changes in the proportions of the various cell types, changes in the specific gene expression of the various cell types, or both. In situations where the pertinent genetic marker is expressed in a subset specific manner, studies based on mixed cell expression data may be limited because differential expression of genes from one cell type will be diluted by RNA from other cell types. Accordingly, although existing methods of gene profiling can provide data regarding whole blood expression profiles, interpreting that data is subject to some ambiguity.

### SUMMARY

The present disclosure generally provides methods for normalizing gene expression profiles against a cellular repertoire, i.e., the different proportions of various cell types in a sample containing multiple cell types. More specifically, the present disclosure encompasses a method for normalizing the gene expression profile against a mixed cell population. Some embodiments include creating a set of reference gene expression profiles for particular cell subsets. In some aspects, the set of reference profiles is created by expression profiling a purified cell type. The purified cell type can be averaged over a population of healthy and/or diseased individuals. The populations can be further grouped by age, gender, and/or ethnicity.

In some embodiments, a gene expression profile specific for a test subject is created. In some implementations, the subject specific profile can be a whole blood gene profile or a white blood cell fraction gene expression profile. Certain embodiments include measuring cell-type fractions in the test subject. In some embodiments, measurement includes a CBC (Complete Blood Count) Test or a blood differential test. Measurement of the cell-type fraction, in some implementations, involves mathematically estimating the cell-type fractions based on choosing the fractions which optimize the fit of the mixture of the canonical profiles to the observed expression profile.

In some embodiments, a transformed expression profile is created by combining the cell-type fraction information with the reference cell expression profiles. The transformed expression profile, in turn, may be used for training or validating disease-classifiers or continuous predictors. In certain aspects, the transformed expression profile can also be used for classifying or predicting the states of the particular subject (e.g., present disease stage). In some embodiments, transformed profile is used for assessing disease risk in the test subject or the probability of a positive or negative response to a drug.

In one aspect, the present disclosure relates to a method for normalizing a gene expression profile of a mixed cell population biological sample of a test subject including obtaining proportion data quantifying a relative proportion of each cell type of a number of cell types within the biological sample of the test subject, where each cell type of the number of cell types corresponds to a respective sub-sample of the biological sample. The method may include obtaining a respective gene expression profile of each sub-sample of the biological sample, and normalizing, by a processor of a computing device, for each sub-sample of the biological sample, the gene expression profile with respect to the proportion data to obtain a normalized gene expression profile of the test subject. The method may include analyzing, by the processor, the normalized gene expression profile of the test subject with respect to a reference gene expression profile, and determining, by the processor, correlation information, where the correlation information represents relative correlation between the normalized gene expression profile and the reference gene expression profile.

In some embodiments in which analyzing the normalized gene expression profile of the test subject comprises evaluating a diagnostic classifier, the diagnostic classifier uses the normalized gene expression profile as input, and provides a diagnostic classification or classification score as output, where a classification score may be an estimated probability that the diagnostic classification applies to the test subject. In certain embodiments, the diagnostic classification is autism, autism spectrum disorder (ASD), typical development (TD), atypical development, delayed development not due to autism spectrum disorder (DD), pervasive development disorder (PDD), atypical autism, Asperger's Disorder, attention deficit disorder (ADD), or attention deficit hyperactivity disorder (ADHD). In certain embodiments, two or more classifications (e.g., selected from the aforementioned classifications) are combined into one diagnostic category that is identified by the diagnostic classifier. In certain embodiments, the diagnostic classifier distinguishes between two or more diagnostic classifications (e.g., the classifier distinguishes between two or more of the aforementioned classifications). For example, in certain embodiments, the diagnostic classifier distinguishes between the classification ASD and DD, as described in U.S. Provisional Patent Application No. 61/800,730, filed Mar. 15, 2013, the text of which is incorporated herein by reference in its entirety.

In some embodiments, analyzing the normalized gene expression profile of the test subject includes evaluating a diagnostic classifier using the normalized gene expression profile of the test subject, where the diagnostic classifier is based at least in part on the reference gene expression profile. Determining the correlation information may include identifying a diagnostic classification or classification score for the test subject using the diagnostic classifier.

In some embodiments, the method includes causing, by the processor, presentation of the correlation information for diagnosis purposes. The mixed cell population biological sample may be a bodily fluid sample. The mixed cell population biological sample may be a blood sample. The mixed cell population biological sample may be a buccal swab sample.

In some embodiments, obtaining the proportion data includes separating each cell type of the mixed cell population biological sample to obtain type-purified sub-samples of the biological sample. Separating may include applying one or more of the following separation methods: flow cytometry, centrifugal sedimentation, magnetic activated cell sorting, drop delay or electrophoretic cell sorting, adhesion-based sorting, and antibody surface capture. Separating may include applying fluorescence-activated cell sorting. Obtaining the respective gene expression profile of each sub-sample of the biological sample may include analyzing each type-purified sub-sample of the biological sample. Analyzing each type-purified sub-sample may include sequencing each type-purified sub-sample. Sequencing may include applying at least one of an RNA-Seq method and a Digital Gene Expression method. Analyzing each type-purified sub-sample may include performing microarray analysis of each type-purified sub-sample.

In some embodiments, obtaining the proportion data includes measuring cell-type fractions of each cell type of the mixed cell population of the biological sample. Measuring cell-type fractions may include applying one or more of the following measurement techniques: Complete Blood Count (CBC) testing and blood differential testing. Obtaining the respective gene expression profile of each sub-sample of the biological sample may include quantifying gene expression data relative to respective proportions of each cell type of the mixed cell population of the biological sample.

In some embodiments, obtaining the respective gene expression profile of each sub-sample of the biological sample includes extracting RNA from each sub-sample, and converting the respective RNA into respective cDNA. Obtaining the respective gene expression profile of each sub-sample may include amplifying the cDNA to increase a quantity of cDNA in at least one sub-sample of the biological sample. The method may include attaching and/or incorporating, for each cDNA sample corresponding to each sub-sample, a respective unique identifier. The unique identifier may include a bar code. Obtaining the respective gene expression profile of each sub-sample of the biological sample may include analyzing each cDNA sample corresponding to each sub-sample. Analyzing each cDNA sample may include sequencing each cDNA sample. Obtaining the gene expression profile may include, for each sub-sample of the biological sample, quantifying one or more of counts per gene, counts per exon, counts per splice, and counts per transcript.

In some embodiments, the reference population includes a disease diagnosis population. The method may include, prior to analyzing the normalized gene expression profile of the test subject with respect to the reference gene expression profile, for each biological sample of the number of mixed cell biological samples of a number of subjects in a reference population: determining, by the processor, for each cell type of the mixed cell population of the respective biological sample, a proportion of a sub-sample corresponding to the respective cell type, accessing a respective gene expression profile, and for each sub-sample of the biological sample, determining, by the processor, a normalized sub-sample gene expression profile, where the respective gene expression profile is normalized relative to the respective proportion of the respective sub-sample. The method may include, for each cell type of the mixed cell population, combining, by the processor, the respective normalized sub-sample gene expression profile for each biological sample of the number of mixed cell biological samples of at least a portion of the reference population to determine the reference gene expression profile. The method may include, prior to combining, grouping the reference population with respect to two or more demographic groups, where the portion of the reference population includes a subset of the number of subjects of the reference population belonging to a first demographic group of the two or more demographic groups. The method may further include, prior to analyzing the normalized gene expression profile of the test subject with respect to the reference gene expression profile, obtaining demographic information regarding the test subject, and selecting, by the processor, the normalized gene expression profile based in part upon the demographic information. The method may include, for each cell type of the mixed cell population of the biological sample, combining, by the processor, the respective proportions of each sub-sample of each biological sample of the number of biological samples to determine a typical proportion profile. The method may include analyzing, by the processor, the proportion data with respect to reference proportion data of the reference population, where determining the correlation information further includes determining correlation between the proportion data and the reference proportion data.

In one aspect, the present disclosure relates to a system including a processor and a memory having instructions stored thereon, where the instructions, when executed by the processor, cause the processor to obtain proportion data quantifying a relative proportion of each cell type of a number of cell types within the biological sample of the test subject, where each cell type of the number of cell types corresponds to a respective sub-sample of the biological sample. The instructions may cause the processor to obtain a respective gene expression profile of each sub-sample of the biological sample, and normalize, for each sub-sample of the biological sample, the gene expression profile with respect to the proportion data to obtain a normalized gene expression profile of the test subject. The instructions may cause the processor to analyze the normalized gene expression profile of the test subject with respect to a reference gene expression profile, and determine correlation information, where the correlation information represents relative correlation between the normalized gene expression profile and the reference gene expression profile.

In one aspect, the present disclosure relates to a non-transitory computer readable medium having instructions stored thereon, where the instructions, when executed by a processor, cause the processor to obtain proportion data quantifying a relative proportion of each cell type of a number of cell types within the biological sample of the test subject, where each cell type of the number of cell types corresponds to a respective sub-sample of the biological sample. The instructions may cause the processor to obtain a respective gene expression profile of each sub-sample of the biological sample, and normalize, for each sub-sample of the biological sample, the gene expression profile with respect to the proportion data to obtain a normalized gene expression profile of the test subject. The instructions may cause the processor to analyze the normalized gene expression profile of the test subject with respect to a reference gene expression profile, and determine correlation information, where the correlation information represents relative correlation between the normalized gene expression profile and the reference gene expression profile.

### BRIEF DESCRIPTION OF THE FIGURES

The foregoing and other objects, aspects, features, and advantages of the present disclosure will become more apparent and better understood by referring to the following description taken in conjunction with the accompanying drawings, in which:

FIGS. 1A through 1C are flow charts of example methods for normalizing gene expression profiles;

FIG. 2 is a schematic flow chart showing a method of classifier signature training and/or use involving the normalization of gene expression profiles of mixed cell population biological samples obtained from a number of subjects within at least one reference population;

FIGS. 3A and 3B are flow charts of example methods for using a reference gene expression profile obtained from a reference population to identify diagnosis information regarding a test subject;

FIG. 4 is an exemplary cloud computing environment 500 for use with the systems and methods described herein, in accordance with an illustrative embodiment; and

FIG. 5 is an example of a computing device 600 and a mobile computing device 650 that can be used to implement the techniques described in this disclosure.

The features and advantages of the present disclosure will become more apparent from the detailed description set forth below when taken in conjunction with the drawings, in which like reference characters identify corresponding elements throughout. In the drawings, like reference numbers generally indicate identical, functionally similar, and/or structurally similar elements.

### DETAILED DESCRIPTION

The present disclosure generally provides methods for normalizing gene expression profiles against a cellular repertoire, i.e., the different proportions of various cell types in a sample containing multiple cell types. More specifically, the present disclosure encompasses a method for normalizing the gene expression profile against a mixed cell population such as a blood sample.

### Expression Normalization

Gene expression on, for example, cDNA, derived from a sample containing multiple cell types such as whole blood, can be expressed as a weighted sum of the expression profiles of the different cell types in the sample, weighted according to their proportions, or fractions, in the population. This is described by the following formula:

E_{i,j}=Σₖ(fⱼₖ*pᵢⱼₖ) (1)

wherein Eᵢⱼ is the expression of the gene i in the individual j, fⱼₖ is the fraction of cells of type k in the blood sample of individual j, and pᵢⱼₖ is the expression of gene i in a pure sample of type k cells from individual j. The sum over k of fⱼₖ is 1.

Information regarding the health or physiological state of the individual may be encoded in the expression profiles of the cell types (pᵢⱼₖ) or in the cell type fractions (fⱼₖ) or both. Variations in the cell fractions may obscure changes in the cell type expression profiles. Conversely, variations in the cell type expression profiles can mask changes in the cell type fractions. The present disclosure contemplates that diagnostically useful information may be maximized by determining both the expression profile of the cell type and its proportion in the sample, rather than just the whole blood expression profile (Eᵢⱼ) which is currently obtained by existing methods of gene expression profiling.

In some implementations, a mixed cell population biological sample is a bodily fluid sample such as cerebrospinal fluid (CSF) or blood. The mixed cell population biological sample, in some implementations, is a tissue sample, such as an organ tissue sample or a buccal swab sample.

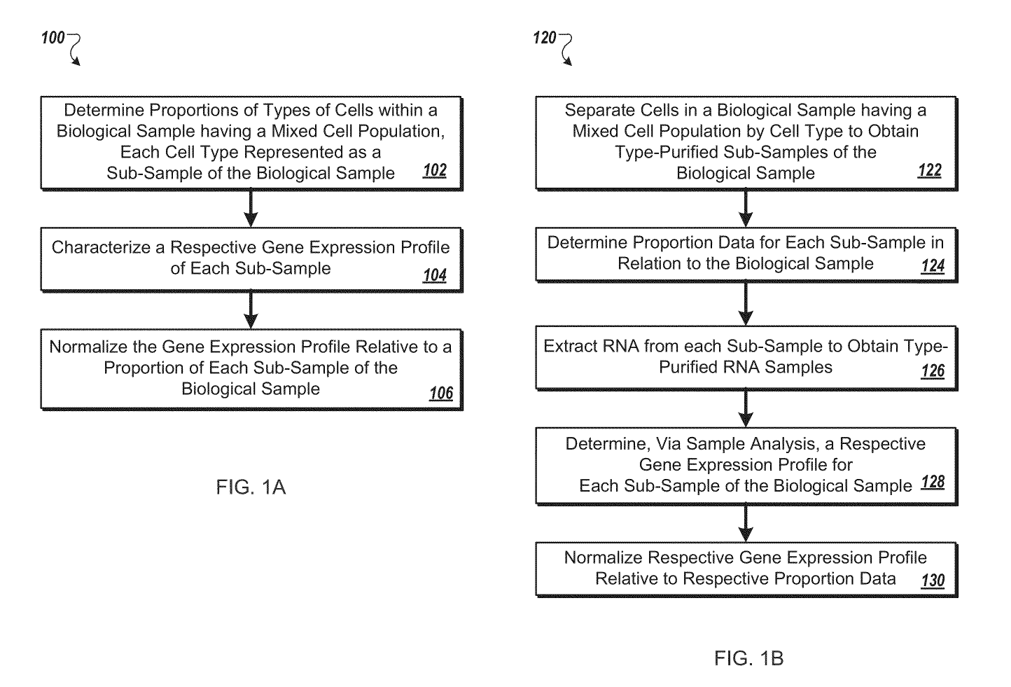

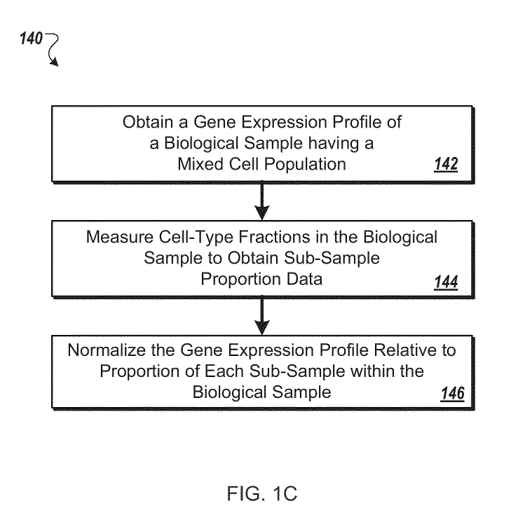

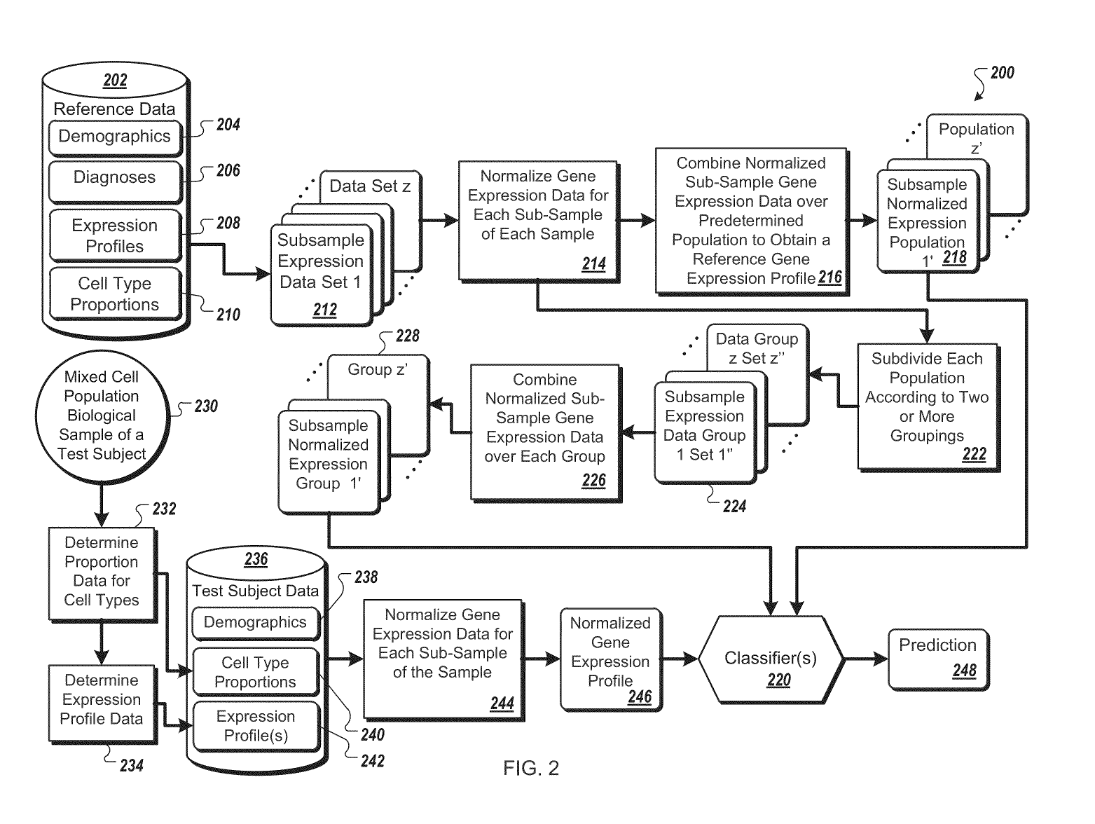

Turning to FIG. 1A, in a general sense, a method 100 for normalizing a gene expression profile for a biological sample having a mixed cell population, in some implementations, begins with determining proportions of types of cells within the biological sample, where each cell type of the mixed cell population corresponds to a respective sub-sample of the biological sample (102). Proportion data, for example, may be determined through physical separation of the individual cell types of the mixed cell population biological sample (e.g., via flow cytometry, etc.) or through measurement of the cell types of the mixed cell population biological sample (e.g., Complete blood Count (CBC) testing, blood differential testing, etc.).

In some implementations, a respective gene expression profile of each sub-sample of the biological sample is characterized (104). If the biological sample has not been physically separated, the biological sample is analyzed (e.g., using a gene sequencer or microarray analysis, etc.). If the sample has been physically separated, in some implementations, each type-purified sub-sample of the biological sample is individually analyzed to determine a respective gene expression profile. For example, the individual type-purified sub-samples may be sequenced using a genomic sequencer or analyzed via microarray analysis. In other implementations, a unique identifier is attached to each type-purified sub-sample such that, upon combination, the cell types are recognizable. The unique identifier may be a DNA bar code. After marking the type-purified sub-samples with a unique identifier, the type-purified sub-samples may be recombined for gene expression analysis (e.g., via sequencing or microarray analysis). The gene expression profile may be obtained, for example, using an RNA-Seq (e.g. Whole Transcriptome Shotgun Sequencing (WTSS)) method and/or a Digital Gene Expression (DGE) profiling method such as serial analysis of gene expression (SAGE). The gene expression profile, in some examples, may include and/or be based on a quantification of counts per gene, counts per exon, counts per splice, and counts per transcript. Counts may refer to reads.

In some implementations, the gene expression profile is normalized relative to a proportion of each sub-sample of the biological sample (106). As described above, gene expression profile data can be expressed as a weighted sum of the expression profiles of the different cell types in the sample, weighted according to their proportions, or fractions, in the population.

Various methods of obtaining additional information regarding cell-type expression profiles along with cell type fractions are contemplated by the present disclosure. This information could be obtained by laboratory methods, including separating cells by cell type prior to expression profiling using flow sorting methods, and profiling each particular subtype separately. This would provide direct measurements of both the expression profile of a particular cell type as well as its proportion in the original population. Additional methods of obtaining information include performing a CBC or blood differential assay on a separate aliquot of blood taken from an individual at the same time that an expression profile sample is taken. The CBC or blood differential assay would not provide information regarding the cell type expression profile, but would provide information regarding the proportion of a cell type in the sample. The information provided by the CBC or blood differential assay can be combined with the gene expression information, as additional gene-like variables in the expression profile, to provide synergistic information when training classifiers or classifying samples.

In a first example, in some implementations, a method 120 for normalizing a gene expression profile for a biological sample having a mixed cell population, in some implementations, begins with separating cells in a biological sample having a mixed cell population by cell type, where each cell type of the mixed cell population is separated out into a separate type-purified sub-sample of the biological sample (122). Separating cells by cell type can be achieved, for example, by one or more cell sorting methods. Various cell sorting methods include flow cytometry, centrifugal sedimentation, magnetic-activated cell sorting, adhesion-based sorting, antibody surface capture, and/or one or more combinations therein.

In some embodiments, cells are sorted by flow cytometry. Flow cytometry is a laser based biophysical technology used in cell counting, cell sorting and biomarker detection, by suspending cells in a stream of fluid and passing them by an electronic detection apparatus. In some embodiments, flow cytometry comprises simultaneous multiparametric analysis of physical and/or chemical characteristics of thousands of cells per second. In some embodiments, cells are physically sorted based on specified properties, so as to purify and/or isolate populations of interest.

In some embodiments, cells are sorted by fluorescence-activated cell sorting (FACS). FACS is a type of flow cytometry which comprises a method for sorting a heterogeneous mixture of cells into two or more subpopulations, one cell at a time, based on the specific light scattering and fluorescent characteristics of each cell. Flow of the cell suspension is adjusted so that there is a large separation between cells relative to their diameter, a vibrating mechanism causes the flow to break into individual droplets, and the system adjusted such that there is a low probability of more than one cell per droplet. Just before the stream breaks into droplets, the flow passes through a fluorescence measuring station where the fluorescent character of interest of each cell is measured. In some embodiments, a charge is placed on the ring based on the immediately prior fluorescence intensity measurement, and the opposite charge is trapped on the droplet as it breaks from the stream. The charged droplets then fall through an electrostatic deflection system that diverts droplets (and individual cells) into containers based upon their charge.

In some embodiments, cells are sorted by centrifugal sedimentation. Centrifugal sedimentation comprises pumping cells into a rotating centrifuge. Cells are separated by specific weight. For example, red blood cells are separated from other cells due to their high specific weight.

In some embodiments, cells are sorted by magnetic-activated cell sorting. In some embodiments, magnetic cell sorting provides a method for isolating cells based upon extracellular properties (e.g. antigens). In some embodiments, magnetic-activated cell sorting comprises magnetic nanoparticles coated with antibodies against a particular surface antigen. In some embodiments, magnetic-activated cell sorting comprises magnetic beads (e.g. dynabeads). In some embodiments, magnetic-activated cell sorting is a column based separation technique where labeled cells are passed through a magnetic column. In some embodiments, magnetic-activated cell sorting comprises a column-free cell separation technique (SEP system) wherein a tube of labeled cells is placed inside a magnetic field; positive cells are retained in the tube while negatively selected cells are in the liquid suspension.

In some implementations, proportion data for each sub-sample in relation to the biological sample can be determined (124). For example, upon physically separating the sample, the type purified sub-samples can be analyzed to determine relative quantities of each cell type. In another example, the biological sample can be analyzed using a measurement technique, such as CBC or blood differential assay, to determine proportion data regarding the biological sample.

In some implementations, RNA is extracted from each type-purified sub-sample (126). The RNA, in some implementations, is converted to cDNA prior to analysis. In some implementations, the RNA (or cDNA) is amplified, for example to increase the quantity of cDNA for analysis purposes.

In some implementations, each sub-sample is analyzed to determine a respective gene expression profile (128). In some implementations, the RNA is analyzed, for example using a sequencing and/or microarray analysis technique. In other implementations, cDNA derived from the RNA is analyzed, for example using a sequencing and/or microarray analysis technique.

In some implementations, respective gene expression profiles of each sub-sample are normalized relative to the respective proportion data (130). As described above, gene expression profile data can be expressed as a weighted sum of the expression profiles of the different cell types in the sample, weighted according to their proportions, or fractions, in the population.

Turning to FIG. 1C, a method 140 illustrates a second example for normalizing a gene expression profile of a mixed cell population biological sample relative to proportion data. In some implementations, the method 140 begins with obtaining a gene expression profile of the biological sample (142). The gene expression profile may be obtained via analysis of the biological sample, for example through sequencing or microarray analysis. In some implementations, RNA is extracted from the biological sample, and the RNA is analyzed. The RNA in some implementations, may be converted to cDNA prior to analysis.

In some implementations, cell-type fractions in the biological sample are measured to obtain sub-sample proportion data (144). The cell-type fractions may be measured, for example, using a measurement technique such as CBC or blood differential assay.

In some implementations, the gene expression profile is normalized relative to proportion of each sub-sample within the biological sample (140). As described above, gene expression profile data can be expressed as a weighted sum of the expression profiles of the different cell types in the sample, weighted according to their proportions, or fractions, in the population.

Computer-based methods may also be used in accordance with various disclosed embodiments to enhance the information content of the expression profiles using cell subtype information. For example, the cell type fraction information could be combined with a set of reference cell type expression profiles to transform the observed data according to the following formula:

Tᵢⱼ=Eᵢⱼ−Σₖ(fⱼₖ*Pᵢⱼₖ) (2)

where Tᵢⱼ is the transformed data and piᵢₖ is the expression of gene i in the canonical profile for cell type k. Least-squares fitting procedures could be used to provide estimates of the cell type fraction (fⱼₖ) given a set of canonical profiles. These estimates in turn enable estimates of the transformed data (Tᵢⱼ). The cell-type data can be added to the transformed data as additional variables to provide an enhanced expression profile. The enhanced expression profile may encode disease-associated information in a more explicit manner, thereby facilitating classifier learning and classification. In particular, the variance of genes may be reduced by removing the variance component attributable to variation in the cell type fractions. If cell type fraction is measured directly by assay, then the measured values can be used in the provided equation rather than least-square estimates.

Reference cell-type expression profiles could be developed by averaging the profiles of flow-sorted purified cells of particular types across a population of healthy individuals. The reference profiles could be matched to the subject by a host of characteristics, such as age, ethnicity, and gender. In some embodiments, pure-subtype profiles of individuals with specific disease conditions are used, as these may provide a better fit to the profile being investigated. In some embodiments, different sets of reference profiles may be compared to determine which one best contributes to the understanding of a subject's blood gene expression. The appropriateness of model fit could be assessed using a variety of statistical methods, such as the sum of the squares of the transformed data divided by a measure of the observed variability of gene i across individuals or within individuals across time.

FIG. 2 is a schematic flow chart showing a method 200 of classifier signature training and/or use involving the normalization of gene expression profiles 208 of mixed cell population biological samples, such as blood samples, obtained from a number of subjects within at least one reference population. Reference populations, for example, may include a healthy (e.g., “normal”) population as well as populations which have been diagnosed with various diseases or other conditions. The gene expression profiles 208 may be divided into separate reference populations with respect to diagnosis data 206.

In some implementations, data regarding subjects within the one or more reference populations is stored within a data store 202 (e.g., digital storage medium such as a computer memory, hard drive, etc.). The data may include demographic data 204, diagnosis data 206, gene expression profiles 208, and cell type proportion data 210.

In some implementations, the method begins with obtaining gene expression profiles 212 for each sub-sample of each subject within one or more reference populations. The gene expression profiles 212, in some implementations, are accessed from the gene expression profiles 208 within the data store 202. In some implementations, the gene expression profiles 212 are derived from the gene expression profiles 208 in light of the corresponding cell type proportion data 210. For example, a sub-sample gene expression profile may be estimated by evaluating whole sample expression data in light of the cell type proportion data.

In some implementations, the gene expression data is normalized for each sub-sample of each sample 212 (214). The gene expression profiles 212, for example, may be normalized using the corresponding cell type proportion data 210.

In some implementations, the normalized sub-sample gene expression profiles may be combined over a predetermined population (e.g., subset of the reference subjects matching in diagnosis data 206) to determine a reference gene expression profile 218 for each predetermined population (216). Combining the gene expression profiles of the predetermined population may include performing mathematical operations such as averaging (e.g., average, median, weighted average, etc.) to identify a “normal” or “typical” gene expression profile pertaining to the predetermined population. In some implementations, one or more sets of outlier gene expression data (e.g., beyond a threshold variance in relation to the majority of the subjects within the predetermined population) may be discarded from consideration in determining the reference gene expression profile.

In some implementations, in addition to combining gene expression profile data, cell type proportion data is combined to determine a “normal” or “typical” proportion (e.g., distribution of cell types) within the predetermined population. The proportion data, for example, can be included within the reference gene expression profile data 218.

The reference gene expression profiles 218 for each predetermined population (e.g., normal, healthy, diagnosed with a disease, diagnosed with a disease in a particular stage, diagnosed with a condition, etc.) may be fed into classifier data 220 to be used in predicting or diagnosing a test subject as to a particular disease or condition.

In some implementations, in addition to or in lieu of creating reference gene expression profiles 218 for particular reference populations, each reference population of the one or more reference populations is subdivided into two or more demographic groupings (222). For example, using the demographic data 204, the reference populations may be subdivided into groups. The groupings, in some examples, may include age ranges, sex, ethnicity, or stage of pregnancy. The sub-sample gene expression profiles, in turn, may be separated into a number of gene expression profile data sets 224 according to both population and demographic group.

In some implementations, the normalized gene expression profile data sets 224 are combined over a predetermined population grouping (e.g., subset of the reference subjects matching in both diagnosis data 206 and demographic data 204) to determine a reference gene expression profile 228 for each predetermined population group (226). Combining the gene expression profiles of the predetermined population group may include performing mathematical operations such as averaging (e.g., average, median, weighted average, etc.) to identify a “normal” or “typical” gene expression profile pertaining to the predetermined population. In some implementations, one or more sets of outlier gene expression data (e.g., beyond a threshold variance in relation to the majority of the subjects within the predetermined population) may be discarded from consideration in determining the reference gene expression profile.

In some implementations, in addition to combining gene expression profile data, cell type proportion data is combined to determine a “normal” or “typical” proportion (e.g., distribution of cell types) within the predetermined population group. The proportion data, for example, can be included within the reference gene expression profile data 228.

The group-specific reference gene expression profiles 228 for each predetermined population may be fed into classifier data 220 to be used in predicting or diagnosing a test subject as to a particular disease or condition.

In order to analyze a test subject in light of the reference gene expression profile data obtained from the subjects within the reference population(s), in some implementations, a mixed cell population biological sample is obtained from a test subject (230). For example, a blood sample, buccal swab sample, spinal fluid sample, or other bodily fluid or organ tissue sample may be obtained from a test subject for analysis and diagnosis purposes.

In some implementations, proportion data 240 pertaining to proportions of types of cells within the biological sample is determined (232). The proportion data 240, for example, may be determined through physical separation of the individual cell types of the mixed cell population biological sample (e.g., via flow cytometry, etc.) or through measurement of the cell types of the mixed cell population biological sample (e.g., Complete blood Count (CBC) testing, blood differential testing, etc.). The proportion data may be stored in a test subject data store 236 for later analysis.

In some implementations, a respective gene expression profile 242 of each sub-sample of the biological sample is characterized (234). If the biological sample has not been physically separated, the biological sample is analyzed (e.g., using a gene sequencer or microarray analysis, etc.). If the sample has been physically separated, in some implementations, each type-purified sub-sample of the biological sample is individually analyzed to determine a respective gene expression profile 242. For example, the individual type-purified sub-samples may be sequenced using a genomic sequencer a or analyzed via microarray analysis. In other implementations, a unique identifier is attached to each type-purified sub-sample such that, upon combination, the cell types are recognizable. The unique identifier may be a DNA bar code. After marking the type-purified sub-samples with a unique identifier, the type-purified sub-samples may be recombined for gene expression analysis (e.g., via sequencing or microarray analysis). The gene expression profile 242 may be obtained, for example, using an RNA-Seq (e.g. Whole Transcriptome Shotgun Sequencing (WTSS)) method and/or a Digital Gene Expression (DGE) profiling method such as serial analysis of gene expression (SAGE). The gene expression profile 242, in some examples, may include and/or be based on a quantification of counts per gene, counts per exon, counts per splice, and counts per transcript. Counts may refer to reads. The gene expression profile 242 may be stored in the test subject data store 236 for later analysis.

In some implementations, the gene expression profile 242 is normalized relative to a proportion of each sub-sample of the biological sample (244). For example, a software-based analysis system may access the proportion data 240 and the gene expression profile(s) 242 to normalize the gene expression profile data 242. As described above, normalized gene expression profile data 246 can be expressed as a weighted sum of the expression profiles of the different cell types in the sample, weighted according to their proportions, or fractions, in the population.

In some implementations, the normalized gene expression profile 246 may be analyzed against one or more reference gene expression profile-based classifiers 220 to determine a prediction 248 related to the test subject. The prediction 248, for example, may include information regarding a relative correlation between the normalized gene expression profile 246 of the test subject and one or more reference gene expression profiles (e.g., population-based and/or group-specific population-based reference gene expression profiles). In some implementations, prediction information is presented to a user, such as a technician or medical professional, for use in diagnosis the test subject in relation to a disease or condition.

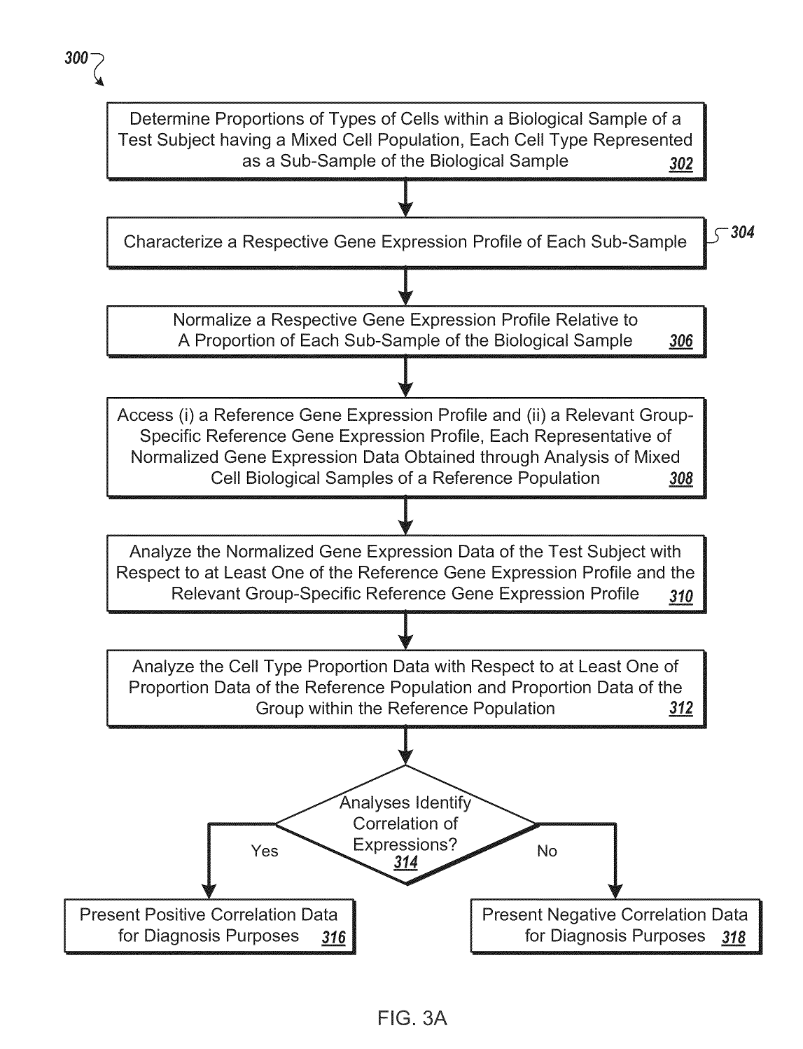

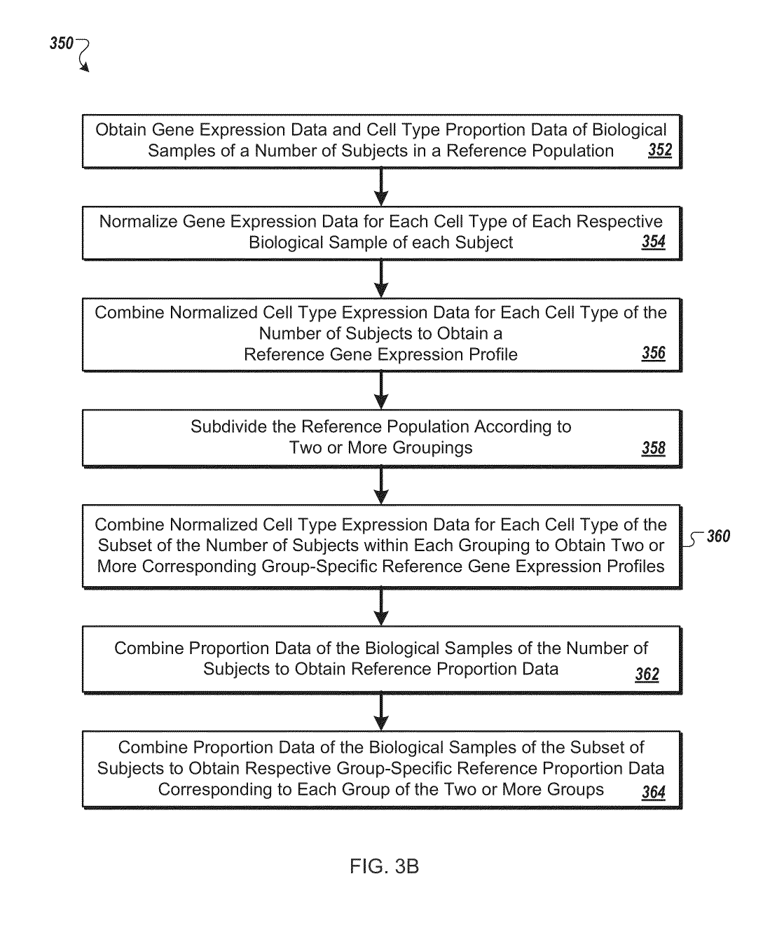

FIGS. 3A and 3B are flow charts of example methods for using a reference gene expression profile obtained from a reference population to identify diagnosis information regarding a test subject.

Turning to FIG. 3A, a method 300 may begin with determining proportions of types of cells within a biological sample of a test subject (302). The biological sample contains a mixed cell population, where each cell type is represented as a sub-sample of the biological sample. Proportion data, for example, may be determined through physical separation of the individual cell types of the mixed cell population biological sample (e.g., via flow cytometry, etc.) or through measurement of the cell types of the mixed cell population biological sample (e.g., Complete blood Count (CBC) testing, blood differential testing, etc.).

In some implementations, a respective gene expression profile may be characterized for each sub-sample (304). If the biological sample has not been physically separated, the biological sample is analyzed (e.g., using a gene sequencer or microarray analysis, etc.). If the sample has been physically separated, in some implementations, each type-purified sub-sample of the biological sample is individually analyzed to determine a respective gene expression profile. For example, the individual type-purified sub-samples may be sequenced using a genomic sequencer a or analyzed via microarray analysis. In other implementations, a unique identifier is attached to each type-purified sub-sample such that, upon combination, the cell types are recognizable. The unique identifier may be a DNA bar code. After marking the type-purified sub-samples with a unique identifier, the type-purified sub-samples may be recombined for gene expression analysis (e.g., via sequencing or microarray analysis). The gene expression profile may be obtained, for example, using an RNA-Seq (e.g. Whole Transcriptome Shotgun Sequencing (WTSS)) method and/or a Digital Gene Expression (DGE) profiling method such as serial analysis of gene expression (SAGE). The gene expression profile, in some examples, may include and/or be based on a quantification of counts per gene, counts per exon, counts per splice, and counts per transcript. Counts may refer to reads.

In some implementations, the respective gene expression profile of each sub-sample may be normalized relative to a proportion of each sub-sample of the biological sample (306). As described above, gene expression profile data can be expressed as a weighted sum of the expression profiles of the different cell types in the sample, weighted according to their proportions, or fractions, in the population.

In some implementations, at least one of a reference gene expression profile and a relevant group-specific reference gene expression profile may be accessed (308). The reference gene expression profile and/or the group-specific reference gene expression profile were obtained through analysis of mixed cell biological samples of a reference population. The reference gene expression profile may be related to a particular population (e.g., normal, healthy, diagnosed with a disease or condition, diagnosed with a particular stage of a disease or condition, etc.). The group-specific reference gene expression profile may be related to a particular demographic group (e.g., age range, sex, ethnicity, stage of pregnancy, etc.) of a particular population. The group-specific reference gene expression profile may be determined to be relevant to the test subject based upon demographic data associated with the test subject. The reference gene expression profile and/or the group specific gene expression profile may be generated by a method 350 described in relation to FIG. 3B. For example, the reference gene expression profile may correspond to one of the reference gene expression profiles 218 described in relation to FIG. 2, while the group-specific reference gene expression profile may correspond to one of the group-specific reference gene expression profiles 228 described in relation to FIG. 2.

In some implementations, the normalized gene expression data of the test subject is analyzed with respect to at least one of the reference gene expression profile and the relevant group-specific reference gene expression profile (310). Gene expression data related to each sub-sample of the test subject, for example, may be evaluated in relation to corresponding gene expression data of the reference. In some implementations, as described in relation to FIG. 2, the reference gene expression profile and/or group specific reference gene expression profile may be combined within a diagnostic classifier.

In some implementations, the cell type proportion data related to the biological sample of the test subject is analyzed with respect to at least one of proportion data of the reference population and proportion data of the group within the reference population (312). For example, a “typical” or “normal” proportion of cell types within the biological samples of the reference population (and/or group thereof) may be accessed and evaluated with respect to the proportions of cell types identified within the biological sample of the test subject.

If the analyses of step 310 and/or step 312 identify a positive correlation between the test subject and reference population (and/or group thereof) (314), in some implementations, positive correlation data is presented for diagnosis purposes (316). If, instead, a negative correlation is determined (314), in some implementations, negative correlation data is presented for diagnosis purposes (318). For example, analysis results may be prepared for presentation upon a display device for review by a user such as a laboratory technician or other medical professional. Correlation outcome, in some implementations, is added to a medical record of the test subject.

Turning to FIG. 3B, the method 350 illustrates example steps that may be included in determining reference gene expression profiles and/or reference proportion data related to one or more reference populations. The reference gene expression profiles and/or reference proportion data obtained through performance of the method 350, for example, may be used in steps 308 and/or 310 the method 300 of FIG. 3A.

In some implementations, the method 350 begins with obtaining gene expression data and cell type proportion data of biological samples of a number of subjects in a reference population (352). For example, the gene expression profile data 208 and the cell type proportion data 210 may be obtained from the data store 202, as described in relation to FIG. 2.

In some implementations, gene expression data for each cell type of each respective biological sample of each subject is normalized in view of the corresponding proportion data (354). As described above, gene expression profile data can be expressed as a weighted sum of the expression profiles of the different cell types in the sample, weighted according to their proportions, or fractions, in the population.

In some implementations, normalized cell type expression data for each cell type of the number of subjects is combined to obtain a reference gene expression profile (356). Combining the gene expression profiles of the predetermined population may include performing mathematical operations such as averaging (e.g., average, median, weighted average, etc.) to identify a “normal” or “typical” gene expression profile pertaining to the predetermined population. In some implementations, one or more sets of outlier gene expression data (e.g., beyond a threshold variance in relation to the majority of the subjects within the predetermined population) may be discarded from consideration in determining the reference gene expression profile.

In some implementations, the reference population is subdivided according to two or more groupings (358). For example, using the demographic data regarding the test subject, such as demographic data 204 described in relation to FIG. 2, the reference populations may be subdivided into groups. The groupings, in some examples, may include age ranges, sex, ethnicity, or stage of pregnancy.

In some implementations, normalized cell type expression data for each cell type of the subset of the number of subjects within each group is combined to obtain two or more group-specific reference gene expression profiles (360). Combining the gene expression profiles of the predetermined population group may include performing mathematical operations such as averaging (e.g., average, median, weighted average, etc.) to identify a “normal” or “typical” gene expression profile pertaining to the predetermined population. In some implementations, one or more sets of outlier gene expression data (e.g., beyond a threshold variance in relation to the majority of the subjects within the predetermined population) may be discarded from consideration in determining the reference gene expression profile.

In some implementations, proportion data of the biological samples of the number of subjects is combined to obtain reference proportion data (362). In some implementations, cell type proportion data is combined to determine a “normal” or “typical” proportion (e.g., distribution of cell types) within the predetermined population.

In some implementations, proportion data of the biological samples of the subset of subjects corresponding to each grouping of the two or more groupings are combined to obtain respective group-specific reference proportion data corresponding to each group of the two or more groups (364). In some implementations, in addition to combining gene expression profile data, cell type proportion data is combined to determine a “normal” or “typical” proportion (e.g., distribution of cell types) within the predetermined population group.

### Expression Analysis

In order to use expression analysis for disorder diagnosis, a threshold of expression is established. The threshold may be established by reference to literature or by using a reference sample from a subject known not to be afflicted with the disorder. The expression profile from the test subject is then compared to the reference threshold. Aberrant expression is an indication that the test subject is afflicted with the disorder. The expression may be over-expression compared to the reference (i.e., an amount greater than the reference) or under-expression compared to the reference (i.e., an amount less than the reference).

Embodiments of the present disclosure may be combined with detection techniques to detect various disorders or risk conditions associated with disorders. For example, aberrant expression (under or over) in the test subject compared to the reference subject indicates that the test subject is afflicted with particular disorder.

Methods of detecting levels of gene products (e.g., RNA or protein) may be used to obtain the expression profile from the target cells. Commonly used methods for the quantification of mRNA expression in a sample include northern blotting and in situ hybridization (Parker & Barnes, Methods in Molecular Biology 106:247 283 (1999); RNAse protection assays (Hod, Biotechniques 13:852 854 (1992; and PCR-based methods, such as reverse transcription polymerase chain reaction (RT-PCR) (Weis et al., Trends in Genetics 8:263 264 (1992). Alternatively, antibodies may be employed that can recognize specific duplexes, including RNA duplexes, DNA-RNA hybrid duplexes, or DNA-protein duplexes. Other example methods for measuring gene expression (e.g., RNA or protein amounts) are described in Yeatman et al. (U.S. patent application number 2006/0195269).

A differentially expressed gene or differential gene expression refer to a gene whose expression is activated to a higher or lower level in a subject suffering from a disorder, relative to its expression in a normal or control subject. The terms also include genes whose expression is activated to a higher or lower level at different stages of the same disorder. It is also understood that a differentially expressed gene may be either activated or inhibited at the nucleic acid level or protein level, or may be subject to alternative splicing to result in a different polypeptide product. Such differences may be evidenced by a change in mRNA levels, surface expression, secretion or other partitioning of a polypeptide, for example. Differential gene expression may include a comparison of expression between two or more genes or their gene products, or a comparison of the ratios of the expression between two or more genes or their gene products, or even a comparison of two differently processed products of the same gene, which differ between normal subjects and subjects suffering from a disorder, such as autism, or between various stages of the same disorder. Differential expression includes both quantitative, as well as qualitative, differences in the temporal or cellular expression profile in a gene or its expression products. Differential gene expression (increases and decreases in expression) is based upon percent or fold changes over expression in normal cells. Increases may be of 1, 5, 10, 20, 30, 40, 50, 60, 70, 80, 90, 100, 120, 140, 160, 180, or 200% relative to expression levels in normal cells. Alternatively, fold increases maybe of 1, 1.5, 2, 2.5, 3, 3.5, 4, 4.5, 5, 5.5, 6, 6.5, 7, 7.5, 8, 8.5, 9, 9.5, or 10 fold over expression levels in normal cells. Decreases may be of 1, 5, 10, 20, 30, 40, 50, 55, 60, 65, 70, 75, 80, 82, 84, 86, 88, 90, 92, 94, 96, 98, 99 or 100% relative to expression levels in normal cells.

In certain embodiments, reverse transcriptase PCR (RT-PCR) is used to measure gene expression. RT-PCR is a quantitative method that can be used to compare mRNA levels in different sample populations to characterize profiles of gene expression, to discriminate between closely related mRNAs, and to analyze RNA structure.

The first step is the isolation of mRNA from a target sample. Methods for mRNA extraction include RNA extraction from paraffin embedded tissues (disclosed, for example, in Rupp and Locker, Lab Invest. 56:A67 (1987), and De Andres et al., BioTechniques 18:42044 (1995)). In particular, RNA isolation can be performed using a purification kit, buffer set and protease from commercial manufacturers, such as Qiagen, according to the manufacturer's instructions. For example, total RNA from cells in culture can be isolated using Qiagen RNeasy mini-columns. Other commercially available RNA isolation kits include MASTERPURE Complete DNA and RNA Purification Kit (EPICENTRE, Madison, Wis.), and Paraffin Block RNA Isolation Kit (Ambion, Inc.). Total RNA from tissue samples can be isolated using RNA Stat-60 (Tel-Test). RNA prepared from tumor can be isolated, for example, by cesium chloride density gradient centrifugation.

The first step in gene expression profiling by RT-PCR is the reverse transcription of the RNA template into eDNA, followed by its exponential amplification in a PCR reaction. The two most commonly used reverse transcriptases are avilo myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived eDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, TaqMan® PCR typically utilizes the 5′-nuclease activity of Taq polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TaqMan® RT-PCR can be performed using commercially available equipment, such as, for example, ABI PRISM 7700™ Sequence Detection System™ (Perkin-Elmer-Applied Biosystems, Foster City, Calif., USA), or Lightcycler (Roche Molecular Biochemicals, Mannheim, Germany). In certain embodiments, the 5′ nuclease procedure is run on a real-time quantitative PCR device such as the ABI PRISM 7700™ Sequence Detection System™. The system consists of a thermocycler, laser, charge-coupled device (CCD), camera and computer. The system amplifies samples in a 96-well format on a thermocycler. During amplification, laser-induced fluorescent signal is collected in real-time through fiber optics cables for all 96 wells, and detected at the CCD. The system includes software for running the instrument and for analyzing the data.

5′-Nuclease assay data are initially expressed as Ct, or the threshold cycle. As discussed above, fluorescence values are recorded during every cycle and represent the amount of product amplified to that point in the amplification reaction. The point when the fluorescent signal is first recorded as statistically significant is the threshold cycle (Ct).

To minimize errors and the effect of sample-to-sample variation, RT-PCR is usually performed using an internal standard. The ideal internal standard is expressed at a constant level among different tissues, and is unaffected by the experimental treatment. RNAs most frequently used to normalize profiles of gene expression are mRNAs for the housekeeping genes glyceraldehyde-3-phosphate-dehydrogenase (GAPDH) and P-actin. For performing analysis on pre-implantation embryos and oocytes, Chuk is a gene that is used for normalization.

A more recent variation of the RT-PCR technique is the real time quantitative PCR, which measures PCR product accumulation through a dual-labeled fluorigenic probe (i.e., TaqMan® probe). Real time PCR is compatible both with quantitative competitive PCR, in which internal competitor for each target sequence is used for normalization, and with quantitative comparative PCR using a normalization gene contained within the sample, or a housekeeping gene for RT-PCR. For further details see, e.g. Held et al., Genome Research 6:986994 (1996).

In another embodiment, a MassARRAY-based gene expression profiling method is used to measure gene expression. In the MassARRAY-based gene expression profiling method, developed by Sequenom, Inc. (San Diego, Calif.) following the isolation of RNA and reverse transcription, the obtained eDNA is spiked with a synthetic DNA molecule (competitor), which matches the targeted eDNA region in all positions, except a single base, and serves as an internal standard. The cDNNcompetitor mixture is PCR amplified and is subjected to a post-PCR shrimp alkaline phosphatase (SAP) enzyme treatment, which results in the dephosphorylation of the remaining nucleotides. After inactivation of the alkaline phosphatase, the PCR products from the competitor and cDNA are subjected to primer extension, which generates distinct mass signals for the competitor- and eDNA-derives PCR products. After purification, these products are dispensed on a chip array, which is pre-loaded with components needed for analysis with matrix- assisted laser desorption ionization time-of-flight mass spectrometry (MALDI-TOF MS) analysis. The eDNA present in the reaction is then quantified by analyzing the ratios of the peak areas in the mass spectrum generated. For further details see, e.g. Ding and Cantor, Proc. Natl. Acad. Sci. USA 100:3059 3064 (2003).

Further PCR-based techniques include, for example, differential display (Liang and Pardee, Science 257:967 971 (1992)); amplified fragment length polymorphism (iAFLP) (Kawamoto et al., Genome Res. 12:1305 1312 (1999)); BeadArray™ technology (Illumina, San Diego, Calif.; Oliphant et al., Discovery of Markers for Disease (Supplement to Biotechniques), I June 2002; Ferguson et al., Analytical Chemistry 72:5618 (2000)); BeadsArray for Detection of Gene Expression (BADGE), using the commercially available Luminex100 LabMAP system and multiple color-coded microspheres (Luminex Corp., Austin, Tex.) in a rapid assay for gene expression (Yang et al., Genome Res. 11:1888 1898 (2001)); and high coverage expression profiling (HiCEP) analysis (Fukumura et al., Nucl. Acids. Res. 31(16) e94 (2003)).

In certain embodiments, differential gene expression can also be identified, or confirmed using a microarray technique. In this method, polynucleotide sequences of interest (including cDNAs and oligonucleotides) are plated, or arrayed, on a microchip substrate. The arrayed sequences are then hybridized with specific DNA probes from cells or tissues of interest. Methods for making microarrays and determining gene product expression (e.g., RNA or protein) are shown in Yeatman et al. (U.S. patent application number 2006/0195269).

In a specific embodiment of the microarray technique, PCR amplified inserts of cDNA clones are applied to a substrate in a dense array, for example, at least 10,000 nucleotide sequences are applied to the substrate. The microarrayed genes, immobilized on the microchip at 10,000 elements each, are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the chip is scanned by confocal laser microscopy or by another detection method, such as a CCD camera. Quantitation of hybridization of each arrayed element allows for assessment of corresponding mRNA abundance. With dual color fluorescence, separately labeled cDNA probes generated from two sources of RNA are hybridized pair-wise to the array. The relative abundance of the transcripts from the two sources corresponding to each specified gene is thus determined simultaneously. The miniaturized scale of the hybridization affords a convenient and rapid evaluation of the expression profile for large numbers of genes. Such methods have been shown to have the sensitivity required to detect rare transcripts, which are expressed at a few copies per cell, and to reproducibly detect at least approximately two-fold differences in the expression levels (see, for example, Schena et al., Proc. Natl. Acad. Sci. USA 93(2):106 149 (1996)). Microarray analysis can be performed by commercially available equipment, following manufacturer's protocols, such as by using the Affymetrix GenChip technology, or Incyte's microarray technology.

Alternatively, protein levels can be determined by constructing an antibody microarray in which binding sites comprise immobilized, preferably monoclonal, antibodies specific to a plurality of protein species encoded by the cell genome. Preferably, antibodies are present for a substantial fraction of the proteins of interest. Methods for making monoclonal antibodies are described, for example, in Harlow and Lane, 1988, ANTIBODIES: A LABORATORY MANUAL, Cold Spring Harbor, N.Y. In one embodiment, monoclonal antibodies are raised against synthetic peptide fragments designed based on genomic sequence of the cell. With such an antibody array, proteins from the cell are expression, and the level of expression, of proteins of diagnostic or prognostic interest can be detected through immunohistochemical staining of tissue slices or sections.

Finally, levels of transcripts of marker genes in a number of tissue specimens may be characterized using a “tissue array” (Kononen et al., Nat. Med 4(7):844-7 (1998). In a tissue array, multiple tissue samples are assessed on the same microarray. The arrays allow in situ detection of RNA and protein levels; consecutive sections allow the analysis of multiple samples simultaneously.

In other embodiments, Serial Analysis of Gene Expression (SAGE) is used to measure gene expression. Serial analysis of gene expression (SAGE) is a method that allows the simultaneous and quantitative analysis of a large number of gene transcripts, without the need of providing an individual hybridization probe for each transcript. First, a short sequence tag (about 10-14 bp) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression profile of any population of transcripts can be quantitatively evaluated by determining the abundance of individual tags, and identifying the gene corresponding to each tag. For more details see, e.g. Velculescu et al., Science 270:484 487 (1995); and Velculescu et al., Cell88:243 51 (1997).

In other embodiments Massively Parallel Signature Sequencing (MPSS) is used to measure gene expression. This method, described by Brenner et al., Nature Biotechnology 18:630 634 (2000), is a sequencing approach that combines non-gel-based signature sequencing with in vitro cloning of millions of templates on separate 5)lm diameter microbeads. First, a microbead library of DNA templates is constructed by in vitro cloning. This is followed by the assembly of a planar array of the template-containing microbeads in a flow cell at a high density (typically greater than 3×106 microbeads/cm2). The free ends of the cloned templates on each microbead are analyzed simultaneously, using a fluorescence-based signature sequencing method that does not require DNA fragment separation. This method has been shown to simultaneously and accurately provide, in a single operation, hundreds of thousands of gene signature sequences from a yeast cDNA library.

Immunohistochemistry methods are also suitable for detecting the expression levels of the gene products of the present disclosure. Thus, antibodies (monoclonal or polyclonal) or antisera, such as polyclonal antisera, specific for each marker are used to detect expression. The antibodies can be detected by direct labeling of the antibodies themselves, for example, with radioactive labels, fluorescent labels, hapten labels such as, biotin, or an enzyme such as horse radish peroxidase or alkaline phosphatase. Alternatively, unlabeled primary antibody is used in conjunction with a labeled secondary antibody, comprising antisera, polyclonal antisera or a monoclonal antibody specific for the primary antibody. Immunohistochemistry protocols and kits are commercially available.

In certain embodiments, a proteomics approach is used to measure gene expression. A proteome refers to the totality of the proteins present in a sample (e.g. tissue, organism, or cell culture) at a certain point of time. Proteomics includes, among other things, study of the global changes of protein expression in a sample (also referred to as expression proteomics). Proteomics typically includes the following steps: (1) separation of individual proteins in a sample by 2-D gel electrophoresis (2-D PAGE); (2) identification of the individual proteins recovered from the gel, e.g. my mass spectrometry or N-terminal sequencing, and (3) analysis of the data using bioinformatics. Proteomics methods are valuable supplements to other methods of gene expression profiling, and can be used, alone or in combination with other methods, to detect the products of the prognostic markers of the present disclosure.

In some embodiments, mass spectrometry (MS) analysis can be used alone or in combination with other methods (e.g., immunoassays or RNA measuring assays) to determine the presence and/or quantity of the one or more biomarkers disclosed herein in a biological sample. In some embodiments, the MS analysis includes matrix-assisted laser desorption/ionization (MALDI) time-of-flight (TOF) MS analysis, such as for example direct-spot MALDI-TOF or liquid chromatography MALDI-TOF mass spectrometry analysis. In some embodiments, the MS analysis comprises electrospray ionization (ESI) MS, such as for example liquid chromatography (LC) ESI-MS. Mass analysis can be accomplished using commercially- available spectrometers. Methods for utilizing MS analysis, including MALDI-TOF MS and ESI-MS, to detect the presence and quantity of biomarker peptides in biological samples are described, for example, in U.S. Pat. Nos. 6,925,389; 6,989,100; and 6,890,763.

### Computer-Implemented Analysis

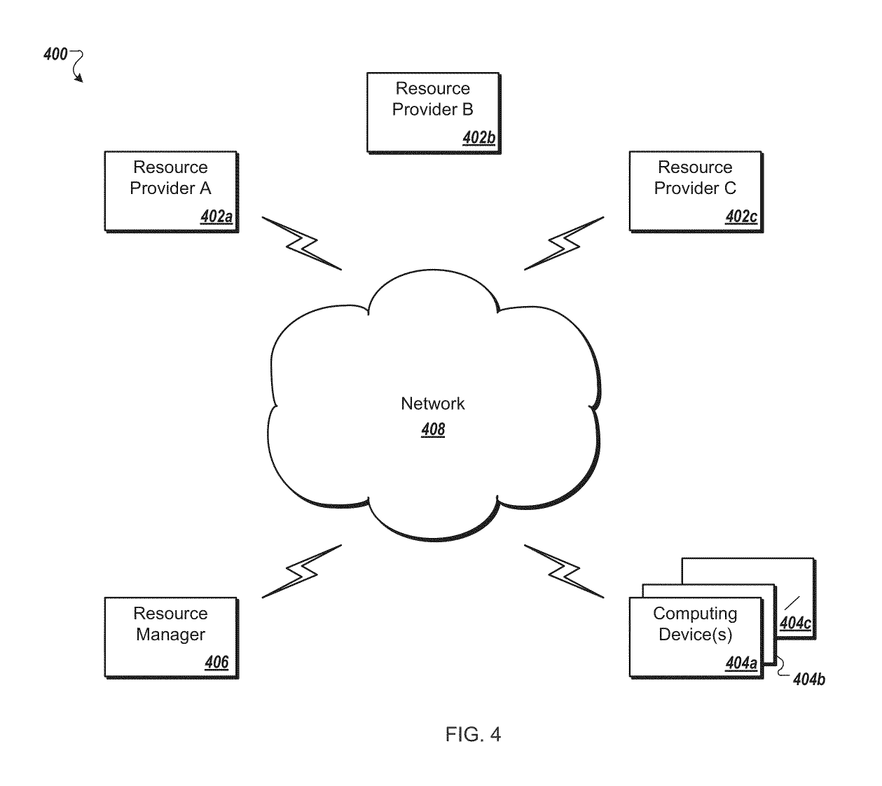

An implementation of an exemplary cloud computing environment 400 for use with the systems and methods described herein is shown in FIG. 4. The cloud computing environment 400 may include one or more resource providers 402a, 402b, 402c (collectively, 402). Each resource provider 402 may include computing resources. In some implementations, computing resources may include any hardware and/or software used to process data. For example, computing resources may include hardware and/or software capable of executing algorithms, computer programs, and/or computer applications. In some implementations, exemplary computing resources may include application servers and/or databases with storage and retrieval capabilities. Each resource provider 402 may be connected to any other resource provider 402 in the cloud computing environment 400. In some implementations, the resource providers 402 may be connected over a computer network 408. Each resource provider 402 may be connected to one or more computing device 404a, 404b, 404c (collectively, 404), over the computer network 408.

The cloud computing environment 400 may include a resource manager 406. The resource manager 406 may be connected to the resource providers 402 and the computing devices 404 over the computer network 408. In some implementations, the resource manager 406 may facilitate the provision of computing resources by one or more resource providers 402 to one or more computing devices 404. The resource manager 406 may receive a request for a computing resource from a particular computing device 404. The resource manager 406 may identify one or more resource providers 402 capable of providing the computing resource requested by the computing device 404. The resource manager 406 may select a resource provider 402 to provide the computing resource. The resource manager 406 may facilitate a connection between the resource provider 402 and a particular computing device 404. In some implementations, the resource manager 406 may establish a connection between a particular resource provider 402 and a particular computing device 404. In some implementations, the resource manager 406 may redirect a particular computing device 404 to a particular resource provider 402 with the requested computing resource.

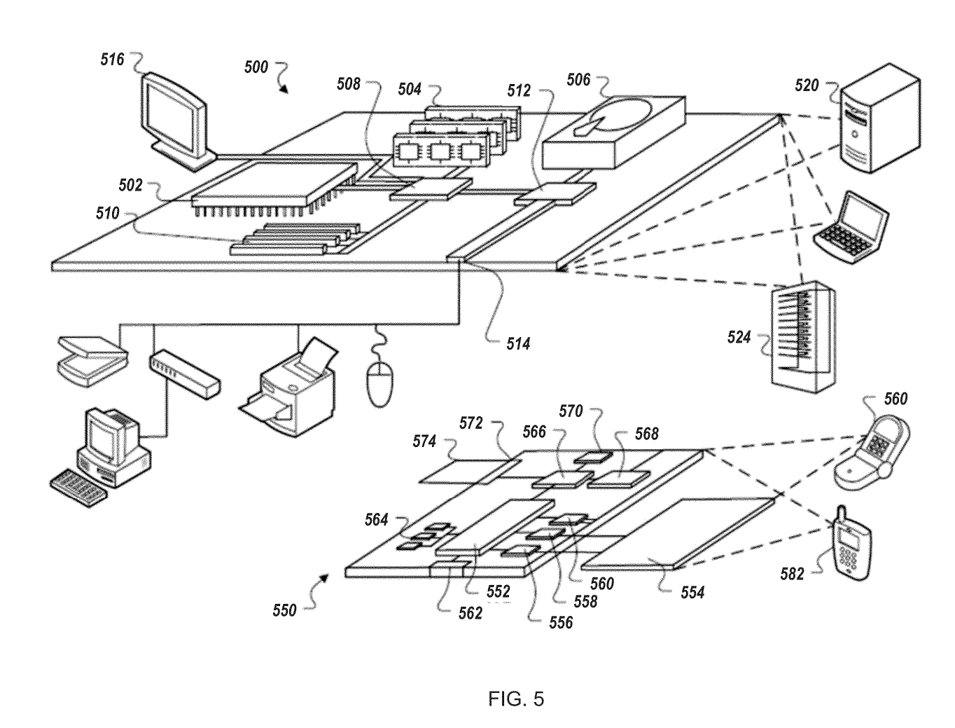

FIG. 5 shows an example of a computing device 500 and a mobile computing device 550 that can be used to implement the techniques described in this disclosure. The computing device 500 is intended to represent various forms of digital computers, such as laptops, desktops, workstations, personal digital assistants, servers, blade servers, mainframes, and other appropriate computers. The mobile computing device 550 is intended to represent various forms of mobile devices, such as personal digital assistants, cellular telephones, smart-phones, and other similar computing devices. The components shown here, their connections and relationships, and their functions, are meant to be examples only, and are not meant to be limiting.

The computing device 500 includes a processor 502, a memory 504, a storage device 506, a high-speed interface 508 connecting to the memory 504 and multiple high-speed expansion ports 510, and a low-speed interface 512 connecting to a low-speed expansion port 514 and the storage device 506. Each of the processor 502, the memory 504, the storage device 506, the high-speed interface 508, the high-speed expansion ports 510, and the low-speed interface 512, are interconnected using various busses, and may be mounted on a common motherboard or in other manners as appropriate. The processor 502 can process instructions for execution within the computing device 500, including instructions stored in the memory 504 or on the storage device 506 to display graphical information for a GUI on an external input/output device, such as a display 516 coupled to the high-speed interface 508. In other implementations, multiple processors and/or multiple buses may be used, as appropriate, along with multiple memories and types of memory. Also, multiple computing devices may be connected, with each device providing portions of the necessary operations (e.g., as a server bank, a group of blade servers, or a multi-processor system).

The memory 504 stores information within the computing device 500. In some implementations, the memory 504 is a volatile memory unit or units. In some implementations, the memory 504 is a non-volatile memory unit or units. The memory 504 may also be another form of computer-readable medium, such as a magnetic or optical disk.

The storage device 506 is capable of providing mass storage for the computing device 500. In some implementations, the storage device 506 may be or contain a computer-readable medium, such as a floppy disk device, a hard disk device, an optical disk device, or a tape device, a flash memory or other similar solid state memory device, or an array of devices, including devices in a storage area network or other configurations. Instructions can be stored in an information carrier. The instructions, when executed by one or more processing devices (for example, processor 502), perform one or more methods, such as those described above. The instructions can also be stored by one or more storage devices such as computer- or machine-readable mediums (for example, the memory 504, the storage device 506, or memory on the processor 502).

The high-speed interface 508 manages bandwidth-intensive operations for the computing device 500, while the low-speed interface 512 manages lower bandwidth-intensive operations. Such allocation of functions is an example only. In some implementations, the high-speed interface 508 is coupled to the memory 504, the display 516 (e.g., through a graphics processor or accelerator), and to the high-speed expansion ports 510, which may accept various expansion cards (not shown). In the implementation, the low-speed interface 512 is coupled to the storage device 506 and the low-speed expansion port 514. The low-speed expansion port 514, which may include various communication ports (e.g., USB, Bluetooth®, Ethernet, wireless Ethernet) may be coupled to one or more input/output devices, such as a keyboard, a pointing device, a scanner, or a networking device such as a switch or router, e.g., through a network adapter.

The computing device 500 may be implemented in a number of different forms, as shown in the figure. For example, it may be implemented as a standard server 520, or multiple times in a group of such servers. In addition, it may be implemented in a personal computer such as a laptop computer 522. It may also be implemented as part of a rack server system 524. Alternatively, components from the computing device 500 may be combined with other components in a mobile device (not shown), such as a mobile computing device 550. Each of such devices may contain one or more of the computing device 500 and the mobile computing device 550, and an entire system may be made up of multiple computing devices communicating with each other.

The mobile computing device 550 includes a processor 552, a memory 564, an input/output device such as a display 554, a communication interface 566, and a transceiver 568, among other components. The mobile computing device 550 may also be provided with a storage device, such as a micro-drive or other device, to provide additional storage. Each of the processor 552, the memory 564, the display 554, the communication interface 566, and the transceiver 568, are interconnected using various buses, and several of the components may be mounted on a common motherboard or in other manners as appropriate.

The processor 552 can execute instructions within the mobile computing device 550, including instructions stored in the memory 564. The processor 552 may be implemented as a chipset of chips that include separate and multiple analog and digital processors. The processor 552 may provide, for example, for coordination of the other components of the mobile computing device 550, such as control of user interfaces, applications run by the mobile computing device 550, and wireless communication by the mobile computing device 550.

The processor 552 may communicate with a user through a control interface 558 and a display interface 556 coupled to the display 554. The display 554 may be, for example, a TFT (Thin-Film-Transistor Liquid Crystal Display) display or an OLED (Organic Light Emitting Diode) display, or other appropriate display technology. The display interface 556 may include appropriate circuitry for driving the display 554 to present graphical and other information to a user. The control interface 558 may receive commands from a user and convert them for submission to the processor 552. In addition, an external interface 562 may provide communication with the processor 552, so as to enable near area communication of the mobile computing device 550 with other devices. The external interface 562 may provide, for example, for wired communication in some implementations, or for wireless communication in other implementations, and multiple interfaces may also be used.

The memory 564 stores information within the mobile computing device 550. The memory 564 can be implemented as one or more of a computer-readable medium or media, a volatile memory unit or units, or a non-volatile memory unit or units. An expansion memory 574 may also be provided and connected to the mobile computing device 550 through an expansion interface 572, which may include, for example, a SIMM (Single In Line Memory Module) card interface. The expansion memory 574 may provide extra storage space for the mobile computing device 550, or may also store applications or other information for the mobile computing device 550. Specifically, the expansion memory 574 may include instructions to carry out or supplement the processes described above, and may include secure information also. Thus, for example, the expansion memory 574 may be provide as a security module for the mobile computing device 550, and may be programmed with instructions that permit secure use of the mobile computing device 550. In addition, secure applications may be provided via the SIMM cards, along with additional information, such as placing identifying information on the SIMM card in a non-hackable manner.

The memory may include, for example, flash memory and/or NVRAM memory (non-volatile random access memory), as discussed below. In some implementations, instructions are stored in an information carrier that the instructions, when executed by one or more processing devices (for example, processor 552), perform one or more methods, such as those described above. The instructions can also be stored by one or more storage devices, such as one or more computer- or machine-readable mediums (for example, the memory 564, the expansion memory 574, or memory on the processor 552). In some implementations, the instructions can be received in a propagated signal, for example, over the transceiver 568 or the external interface 562.

The mobile computing device 550 may communicate wirelessly through the communication interface 566, which may include digital signal processing circuitry where necessary. The communication interface 566 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 568 using a radio-frequency. In addition, short-range communication may occur, such as using a Bluetooth®, Wi-Fi™, or other such transceiver (not shown). In addition, a GPS (Global Positioning System) receiver module 570 may provide additional navigation- and location-related wireless data to the mobile computing device 550, which may be used as appropriate by applications running on the mobile computing device 550.

The mobile computing device 550 may also communicate audibly using an audio codec 560, which may receive spoken information from a user and convert it to usable digital information. The audio codec 560 may likewise generate audible sound for a user, such as through a speaker, e.g., in a handset of the mobile computing device 550. Such sound may include sound from voice telephone calls, may include recorded sound (e.g., voice messages, music files, etc.) and may also include sound generated by applications operating on the mobile computing device 550.

The mobile computing device 550 may be implemented in a number of different forms, as shown in the figure. For example, it may be implemented as a cellular telephone 580. It may also be implemented as part of a smart-phone 582, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

In view of the structure, functions and apparatus of the systems and methods described here, in some implementations, a systems, methods, and apparatus for normalizing gene expression profiles against a cellular repertoire are provided. Having described certain implementations of methods, systems, and apparatus herein, it will now become apparent to one of skill in the art that other implementations incorporating the concepts of the disclosure may be used. Therefore, the disclosure should not be limited to certain implementations, but rather should be limited only by the spirit and scope of the following claims.

What is claimed is:

1. A method for normalizing a gene expression profile of a mixed cell population biological sample of a test subject, the method comprising:
obtaining proportion data quantifying a relative proportion of each cell type of a plurality of cell types within the biological sample of the test subject, wherein each cell type of the plurality of cell types corresponds to a respective sub-sample of the biological sample;
obtaining a respective gene expression profile of each sub-sample of the biological sample;
normalizing, by a processor of a computing device, for each sub-sample of the biological sample, the gene expression profile with respect to the proportion data to obtain a normalized gene expression profile of the test subject;
analyzing, by the processor, the normalized gene expression profile of the test subject with respect to a reference gene expression profile; and
determining, by the processor, correlation information, wherein the correlation information represents relative correlation between the normalized gene expression profile and the reference gene expression profile.

2. The method of claim 1, wherein analyzing the normalized gene expression profile of the test subject comprises evaluating a diagnostic classifier using the normalized gene expression profile of the test subject, wherein the diagnostic classifier is based at least in part on the reference gene expression profile.

3. The method of claim 2, wherein determining the correlation information comprises identifying a diagnostic classification or classification score for the test subject using the diagnostic classifier.

4. The method of claim 1, further comprising causing, by the processor, presentation of the correlation information for diagnosis purposes.

5. The method of claim 1, wherein the mixed cell population biological sample is a bodily fluid sample.

6. The method of claim 5, wherein the mixed cell population biological sample is a blood sample.

7. The method of claim 1, wherein the mixed cell population biological sample is a buccal swab sample.

8. The method of claim 1, wherein obtaining the proportion data comprises separating each cell type of the mixed cell population biological sample to obtain type-purified sub-samples of the biological sample.

9. The method of claim 8, wherein separating comprises applying one or more of the following separation methods: flow cytometry, centrifugal sedimentation, magnetic activated cell sorting, drop delay or electrophoretic cell sorting, adhesion-based sorting, and antibody surface capture.

10. The method of claim 9, wherein separating comprises applying fluorescence-activated cell sorting.

11. The method of claim 8, wherein obtaining the respective gene expression profile of each sub-sample of the biological sample comprises analyzing each type-purified sub-sample of the biological sample.

12. The method of claim 11, wherein analyzing each type-purified sub-sample comprises sequencing each type-purified sub-sample.

13. The method of claim 12, wherein sequencing comprises applying at least one of an RNA-Seq method and a Digital Gene Expression method.

14. The method of claim 11, wherein analyzing each type-purified sub-sample comprises performing microarray analysis of each type-purified sub-sample.

15. The method of claim 1, wherein obtaining the proportion data comprises measuring cell-type fractions of each cell type of the mixed cell population of the biological sample.

16. The method of claim 15, wherein measuring cell-type fractions comprises applying one or more of the following measurement techniques: Complete Blood Count (CBC) testing and blood differential testing.

17. The method of claim 15, wherein obtaining the respective gene expression profile of each sub-sample of the biological sample comprises quantifying gene expression data relative to respective proportions of each cell type of the mixed cell population of the biological sample.

18. The method of claim 1, wherein obtaining the respective gene expression profile of each sub-sample of the biological sample comprises extracting RNA from each sub-sample, and converting the respective RNA into respective cDNA.

19. The method of claim 18, wherein obtaining the respective gene expression profile of each sub-sample comprises amplifying the cDNA to increase a quantity of cDNA in at least one sub-sample of the biological sample.

20. The method of claim 18, further comprising attaching and/or incorporating, for each cDNA sample corresponding to each sub-sample, a respective unique identifier.

21. The method of claim 20, wherein the unique identifier comprises a bar code.

22. The method of claim 18, wherein obtaining the respective gene expression profile of each sub-sample of the biological sample comprises analyzing each cDNA sample corresponding to each sub-sample.

23. The method of claim 22, wherein analyzing each cDNA sample comprises sequencing each cDNA sample.

24. The method of claim 22, wherein obtaining the gene expression profile comprises, for each sub-sample of the biological sample, quantifying one or more of counts per gene, counts per exon, counts per splice, and counts per transcript.

25. The method of claim 1, wherein the reference population comprises a disease diagnosis population.

26. The method of claim 1, further comprising, prior to analyzing the normalized gene expression profile of the test subject with respect to the reference gene expression profile:
for each biological sample of the plurality of mixed cell biological samples of a plurality of subjects in a reference population:
determining, by the processor, for each cell type of the mixed cell population of the respective biological sample, a proportion of a sub-sample corresponding to the respective cell type,
accessing a respective gene expression profile, and
for each sub-sample of the biological sample, determining, by the processor, a normalized sub-sample gene expression profile, wherein the respective gene expression profile is normalized relative to the respective proportion of the respective sub-sample; and
for each cell type of the mixed cell population, combining, by the processor, the respective normalized sub-sample gene expression profile for each biological sample of the plurality of mixed cell biological samples of at least a portion of the reference population to determine the reference gene expression profile.

27. The method of claim 26, further comprising:
prior to combining, grouping the reference population with respect to two or more demographic groups, wherein the portion of the reference population comprises a subset of the plurality of subjects of the reference population belonging to a first demographic group of the two or more demographic groups.

28. The method of claim 27, further comprising, prior to analyzing the normalized gene expression profile of the test subject with respect to the reference gene expression profile:
obtaining demographic information regarding the test subject; and
selecting, by the processor, the normalized gene expression profile based in part upon the demographic information.

29. The method of claim 26, further comprising, for each cell type of the mixed cell population of the biological sample, combining, by the processor, the respective proportions of each sub-sample of each biological sample of the plurality of biological samples to determine a typical proportion profile.

30. The method of claim 29, further comprising analyzing, by the processor, the proportion data with respect to reference proportion data of the reference population, wherein determining the correlation information further comprises determining correlation between the proportion data and the reference proportion data.

31. A system for normalizing a gene expression profile of a mixed cell population biological sample of a test subject, the system comprising:
a processor; and
a memory having instructions stored thereon, wherein the instructions, when executed by the processor, cause the processor to:
obtain proportion data quantifying a relative proportion of each cell type of a plurality of cell types within the biological sample of the test subject, wherein each cell type of the plurality of cell types corresponds to a respective sub-sample of the biological sample;
obtain a respective gene expression profile of each sub-sample of the biological sample;
normalize, for each sub-sample of the biological sample, the gene expression profile with respect to the proportion data to obtain a normalized gene expression profile of the test subject;
analyze the normalized gene expression profile of the test subject with respect to a reference gene expression profile; and
determine correlation information, wherein the correlation information represents relative correlation between the normalized gene expression profile and the reference gene expression profile.

32. A non-transitory computer readable medium having instructions stored thereon, wherein the instructions, when executed by a processor, cause the processor to:
obtain proportion data quantifying a relative proportion of each cell type of a plurality of cell types within a biological sample of a test subject, wherein each cell type of the plurality of cell types corresponds to a respective sub-sample of the biological sample;
obtain a respective gene expression profile of each sub-sample of the biological sample;
normalize, for each sub-sample of the biological sample, the gene expression profile with respect to the proportion data to obtain a normalized gene expression profile of the test subject;
analyze the normalized gene expression profile of the test subject with respect to a reference gene expression profile; and
determine correlation information, wherein the correlation information represents relative correlation between the normalized gene expression profile and the reference gene expression profile.
